(19) United States
(12) Patent Application Publication
(10) Pub. No.: US 2010/0063376 A1
(43) Pub. Date: Mar. 11, 2010

(54) NERVE MONITORING DEVICE

(76) Inventor: Jack M. Kartush, Farmington Hills, MI (US)

Correspondence Address: WARNER NORCROSS & JUDD LLP, 900 FIFTH THIRD CENTER, 111 LYON STREET, N.W., GRAND RAPIDS, MI, 49503-2487, US

(21) Appl. No.: 12/523,931

(22) PCT Filed: Jan. 23, 2008
(86) PCT No.: PCT/US08/51768

§ 371 Date: Jul. 21, 2009

Related U.S. Application Data
(60) Provisional application No. 60/886,119, filed on Jan. 23, 2007.

(51) Int. Cl. A61B 5/04

(52) U.S. Cl. 600/380

(57) ABSTRACT

The present invention provides a nerve monitoring device. The device includes a cannula, a sensor for monitoring the nerve and an alignment device. The cannula can be any surgical cannula, and is preferably an endotracheal tube. The sensor can be an electrode or other sensor that is capable of sensing nerve activity. The alignment device is a device that ensures that after insertion of the nerve sensor into a patient, the sensors are aligned to properly monitor the target nerve or muscle. The internal alignment device may communicate externally to surgeon by using electromagnetic energy as either a transmittor or a receiver. The mismatch of triangular laryngeal anatomy to circular cannula anatomy can be compensated for by a) altering the geometry (external shape) of the cannula and b) using soft, felt-like expandable electrodes. Rotational error can be compensated for by using a multi-electrode array wherein the optimized recording montage can be simply selected on the external recording device.

### BACKGROUND OF THE INVENTION

1. Technical Field

The present invention relates to nerve monitoring. More specifically, the present invention relates to a device to assist in nerve monitoring.

2. Description of the Related Art

A, serious problem for surgeons is avoiding the risk of vocal cord paralysis following thyroid, parathyroid and skull base surgery. The small and difficult to find Recurrent Laryngeal Nerves may be inadvertently injured by even the most experienced surgeon. Simply trying to identify the nerves can stretch or tear the nerve resulting in hoarseness, difficulty with speech, aspiration of food and liquids that can result in pneumonia, as well as life-threatening airway obstruction. Consequently, intraoperative nerve monitoring techniques initially used in ear, brain and spine surgery are being applied today to reduce the risk of vocal cord paralysis.

Monitoring of the facial nerve during acoustic tumor surgery has become a model of how intraoperative neurophysiologic testing can help locate and preserve cranial nerves. Kartush J M: Electroneurography and Intraoperative Facial Monitoring in Contemporary Neurotology. Otolaryngology-Head and Neck Surgery, Vol. 101, No. 4, pp. 496-503, October, 1989; Kartush J, Prass R: Facial nerve testing: ENoG and intraoperative monitoring, J. Johnson (ed.). Mosby, 1988; Kartush J M, Niparko J K, et al: Intraoperative Facial Nerve Monitoring: A Comparison of Stimulating Electrodes. Laryngoscope, Vol. 95, pp. 1536-1540, December, 1985. Kartush J M, Lundy L: Facial Nerve Outcome in Acoustic Neuroma Surgery. Otolaryngologic Clinics of North America, Vol. 25, No. 3, pp. 623-647, June, 1992; Kartush J M, Bouchard K R: Intraoperative Facial Nerve Monitoring: Otology, Neurotology and Skull Base Surgery. Neuromonitoring in Otology and Head and Neck Surgery. J. M. Kartush, K. R. Bouchard (eds.). Raven Press, New York, ch. 5, pp. 99-120, 1992; Kartush J M, Brackmann D E: Acoustic Neuroma Update. Otolaryngologic Clinics of North America. Jack M Kartush, MD (ed.) W. B. Saunders, Philadelphia, vol 29, number 3, June 1996. In contrast, the benefits of recurrent laryngeal nerve (RLN) monitoring during thyroidectomy and parathyroidectomy are much more modest. Although the literature demonstrates favorable experiences, there are also a plethora of publications that find little or no benefit of RLN monitoring. One representative paper entitled “Recurrent Laryngeal Nerve Electrophysiologic Monitoring in Thyroid Surgery: The Standard of Care?” concluded “Electrophysiologic RLN monitoring was not demonstrated in this study to reduce the incidence of transient or permanent vocal fold immobility after thyroid surgery. Electrophysiologic RLN integrity does not always translate into clinical postoperative vocal fold mobility”. Robert L. Witt: Recurrent Laryngeal Nerve Electrophysiologic Monitoring in Thyroid Surgery: The Standard of Care? Journal of Voice, Volume 19, Issue 3, September 2005, Pages 497-500

There are a variety of reasons that may account for the apparent low benefit of RLN monitoring. For example, the low incidence of complications in experienced hands requires large sample sizes to show significant benefit. Also, most published articles are by experienced surgeons with lower than average complication rates. Current forms of RLN monitoring may also be inaccurate or ineffective due to anatomic, physiologic and technical causes.

Consequently, there is a need to identify the numerous sources of inefficiency and error in laryngeal monitoring compared to other types of monitoring. A comparison to the extremely reliable and accurate modality of facial nerve monitoring is helpful in determining how RLN monitoring can be improved. The intracranial portion of the facial nerve is the main trunk of the nerve which is unmyelinated making it exquisitely sensitive to both electrical and mechanical stimulation. The RLN, however, is a small, myelinated branch of the vagus nerve resulting in reduced sensitivity to electrical and, in particular, mechanical stimulation. Direct recording from the large facial muscles with intramuscular needle electrodes readily allows detection of a robust EMG response. In contrast, monitoring of the small laryngeal muscles typically employs surface electrodes due to the practical difficulty and risks associated with placement of needle electrodes in the delicate laryngeal muscles. Placing surface electrodes on an endotracheal tube (ET tube) provides a practical method of obtaining proximity of electrodes to vocal cords. However, this expediency carries significant disadvantages. Detection of the EMG response is compromised not only by the inherent diminished amplitude of surface recording but due to difficulties in ensuring optimal contact between electrode and vocal cord.

The ability to optimize the Electrode-Vocal Cord (EVC) contact is limited by a number of factors. First, direct visualization of the EVC juxtaposition typically occurs only during intubation. Even if it is transiently checked once again after positioning the patient, loss of optimal EVC contact may go undetected. Furthermore, anterior location of the larynx or a large, floppy epiglottis can prevent direct visualization even with a laryngoscope. Although this could be overcome by a flexible scope, the time and expense to add flexible fiberoptic endoscopy following standard intubation with a rigid laryngoscope makes it impractical if not prohibitive.

Second, there are numerous causes of electrode malposition. It can be caused by too small of an ET, which would prevent adequate EVC contact. One company, Medtronic (MDT) has attempted to minimize this by making tubes larger than normal, but this can make intubation more difficult and may cause pressure trauma to the vocal cords. The company's lack of “half size” tubes, exacerbates this problem. Other causes include the anatomic variances within the pharynx and larynx that may force the tube to enter the glottis at an angle that reduces contact at the EVC interface i.e. ET too anterior or too posterior. Also, too deep or too shallow insertion of the ET displaces the electrodes inferior or superior to the vocal cords. Rotation of the ET skews the electrodes away from the vocal cords, which can result in a false negative error. The recent change to a more rigid reinforced tube (intended to make intubation easier) exacerbates the problem as minor rotations of the tube at the mouth can result in rotations at the vocal cords. To compensate for inaccurate tube insertion depth, current iterations of the commercial tube have incorporated an increase in the uninsulated contact area of the electrodes. This modification, however, increases the possibility of false positive error i.e. inadvertent electric stimulation of the inferior constrictor muscle may be misinterpreted as true vocal cord movement because the increased exposure of the tube's electrodes will pick up inferior constrictor muscle activity.

The third problem is drying at the EVC interface increases impedance which reduces detectability of the EMG response. And fourth, too much moisture from secretions or intentionally applied lubricating jelly may cause shunting of the electrical response away from the electrodes.

Sub-optimal recoding parameters also create both false positive and false negative errors. For example if the stimulus filter (Ignore Period) is set too long, it may filter out both the true response as well as the stimulus artifact.

The reduced responsiveness of the RLN compared to the facial nerve, means that the surgeon cannot rely on mechanical evoked potentials, as is commonly done during brain surgery. Therefore, frequent electric stimulation using instruments such as the Kartush Stimulating Dissection Instruments (KSDs) [Jack, we need a generic name for this so the examiner knows what we are referring to.] allow ongoing mapping of nerve location. Education is required of thyroid surgeons to assure frequent Stimulating Dissection as well as avoidance of cautery near the nerves because the Monitor cannot function during cautery.

There are two major monitoring techniques that have been advocated for electromyography (EMG) to enhance laryngeal nerve preservation. They are classified as invasive (needle) or noninvasive (surface) electrodes.

Indwelling needle electrodes allow the most precise measure of the small electrical changes that occur when the laryngeal muscles have been stimulated mechanically or electrically. This technique suffers from two drawbacks: a) injury of the delicate vocal cord muscles by the penetrating needles, and b) difficulty in visualizing and accessing the cords. For example, puncturing the laryngeal muscles with needle electrodes can result in bleeding, scarring and infection.

Because of the deep, relatively inaccessible location of the vocal cords in the throat, needle insertion has typically required the expertise of an Ear Nose and Throat doctor (otolaryngologist) using an endoscope. Accurate placement of the needles through a long scope into tiny muscles is nonetheless a difficult endeavor. Furthermore, most thyroid and parathyroid operations have been performed by General Surgeons who have little or no training in laryngeal endoscopy.

Consequently, an alternate technique has been an external approach to open the neck incision and then penetrate the laryngeal muscles or cricothyroideus membrane from outside to reach the internal vocal cords. This method has fewer drawbacks than the direct endoscopic approach but still requires considerable skill since the electrodes are placed blindly from outside to in, and the final electrode position cannot be visually confirmed.

Another problem is that simple needle electrodes may become displaced during surgery. While hook-shaped wire electrodes are more secure, they may cause more injury when they are later withdrawn.

These practical drawbacks of invasive needle placement have led to the burgeoning use of non-invasive surface contacts. Because most thyroid operations are performed under general anesthesia with an ET tube inserted by the anesthesiologist to assist in respiratory ventilation, it is expedient to place an electrode on the tube and have it rest adjacent to the cords. The challenge here, as detailed above, is to avoid inadequate Electrode-Vocal Cord (EVC) contact.

There are two commercial surface electrodes for laryngeal monitoring, the Medtronic integrated ET tube electrode, with two pairs of bare wires facing each vocal cord, and the Neurovision Medical Products attachment ET tube electrode (U.S. Pat. No. 5,178,145 issued to Rea) with a single electrode plate facing each vocal cord. The ET tube-borne electrodes can be not only difficult to accurately place, but difficult to maintain in proper position.

Another option is to use a surface EMG electrode in the postcricoid location. In this case the electrode is attached to a soft paddle and placed by laryngoscopy behind the larynx adjacent to the posterior cricoarytenoideus muscles. This monitors the largest muscles of the larynx, and the only pure abductors. Similar to the case of the ET tube electrode, the postcricoid placement requires considerable experience and skill to properly place the device—but rarely is such expertise available.

One problem with laryngeal surface electrodes is that the aperture created by the human glottis is triangular whereas the ET tube is round. This creates a fundamental mismatch between the surfaces. Ideally a surface electrode should be conformational to the surface being monitored. Attempts to improve the Electrode-Vocal Cord contact by simply increasing the outer diameter of standard tubes to put more pressure of the electrode onto the vocal cords can lead to difficult and traumatic intubations as well as the possibility of pressure-induced vocal cord injury, particularly during prolonged operations such as removal of skull base tumors.

A second problem is rotation of the ET tube around its long axis which displaces the electrodes away from the cords.

A third problem is the depth that the ET tube is inserted. Similar to rotation, a ET tube placed too shallow or too deep within the throat will result in poor electrode contact with the cords. A ET tube inserted too deep may not only miss the cords but may pick up activity from other lower muscles in the neck (pharyngeal constrictors). Such “false positive errors’ can lead to considerable anatomic disorientation of the surgeon.

Once the ET tube is in the patient's throat, the ET tube cannot normally be seen. Thus if the patient's head is subsequently moved after intubation, as typically occurs with surgical positioning, even a properly placed ET tube may become dislocated. Attempts to once again verify position of the ET tube even with newer technology rigid and flexible endoscopes can be confounded by the patient's anatomy (a floppy epiglottis, a large tongue), saliva, fogging of the endoscope lens, etc. Thus, an innovation is required to essentially “ping” the device and assure proper placement at and after intubation, while allowing safe, maximized Electrode-Vocal Cord contact.

### SUMMARY OF THE INVENTION

The present invention provides a nerve monitoring device. The device includes a cannula, a sensor for monitoring the nerve and an alignment device. The cannula can be any surgical cannula, and is preferably an ET tube. The sensor can be an electrode or other sensor that is capable of sensing nerve or muscle activity. The alignment device is a device that ensures that after insertion of the sensor into a patient, the sensors are aligned to properly monitor the target nerve or muscle. The internal alignment device may communicate externally to a surgeon by using electromagnetic energy as either a transmitter or a receiver to convey information on ET tube depth and rotational alignment. The mismatch of triangular laryngeal anatomy to circular cannula anatomy can be compensated for by a) altering the geometry (external shape) of the cannula and b) using soft, felt-like expandable electrodes in lieu of the conventional non-yielding metal electrodes. Rotational error can be compensated for by using a multi-electrode array wherein the optimized recording montage can be simply selected on the external recording device.

The nerve monitor can be inserted into a patient at a desired location in order to monitor the activity of nerve or muscle. Once inserted the monitor is attached to a device that can analyze the output of the monitor and provide information with regard to the nerve activity.

These and other objects, advantages and features of the invention will be more fully understood and appreciated by reference to the description of the current embodiment and the drawings.

### BRIEF DESCRIPTION OF THE DRAWINGS

FIGS. 1A-C show prior art devices (FIG. 1A and FIG. 1B) and the nerve monitor of the present invention in place in a patient (FIG. 1C).

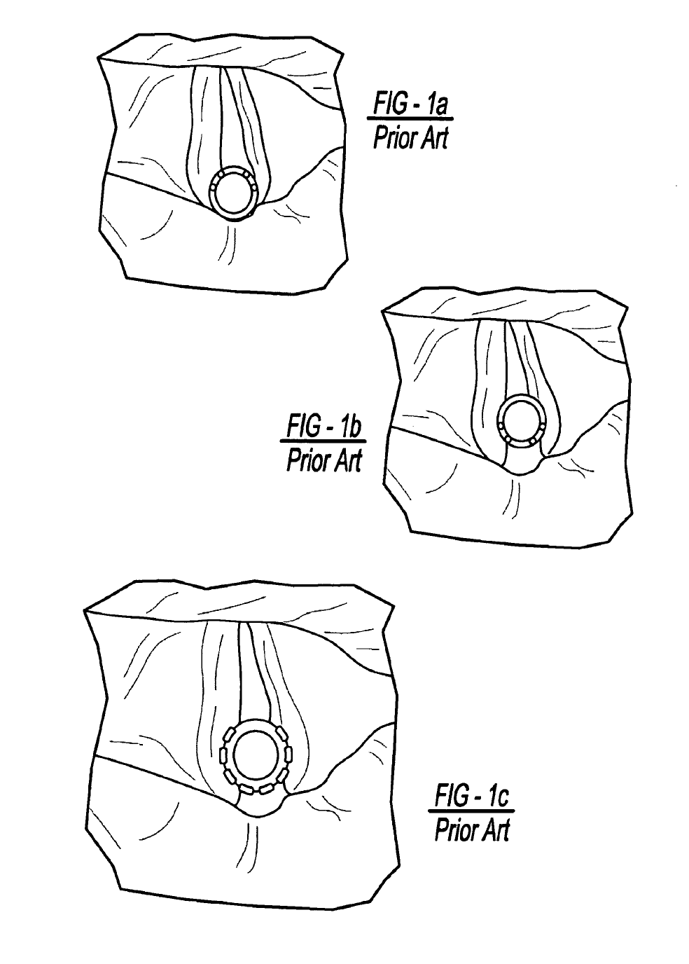

FIG. 2 is a side view of the ET tube of the present invention in use.

### DESCRIPTION OF THE CURRENT EMBODIMENT

Generally, the present invention provides a device for monitoring nerves to detect nerve or muscle activity. The device is generally shown as 10 in the drawings and includes a cannula 12 and at least one sensing device 14.

The cannula 12 can be any device known to those of skill in the art as being insertable into a patient. The cannula 12 is made of a biocompatible material that is either disposable or sterilizable. The cannula 12 can be formed of a plastic and can include a coating on the exterior surface 13. For example, the coating can be used to enable easier insertion of the cannula 12, or can include a material that limits or prevents an adverse reaction in the patient after insertion of the cannula 12.

The cannula 12 can be an endotracheal tube 12′, as shown in the figures. The “endotracheal tube” of the present invention can be any endotracheal tube 12′ known to those of skill in the art. An endotracheal tube 12′ (also called an ET tube or ETT) is used in anaesthesia, intensive care and emergency medicine for airway management and mechanical ventilation. The ET tube 12′ is inserted into a patient's trachea in order to ensure that the airway is not closed off and that air is able to reach the lungs. The ET tube 12′ is regarded as the most reliable available method for protecting a patient's airway.

There are many types of ET tubes, ET tubes range in size from 3-10.5 mm in internal diameter (ID)—different sizes are chosen based on the patient's body size with the smaller sizes being used for paediatric and neonatal patients. ET tubes larger than 6 mm ID tend to have an inflatable cuff. While the present invention is discussed in terms of an ET tube, other cannulas can also be developed that can include similar sensors for monitor different nerve activity. The device can also be applied to a conventional ET tube.

The “sensors” of the present invention can be any sensor that is able to detect nerve activity. Examples of such sensors 14 include electrodes and chemical sensors. The chemical sensors can be sensors that detect an increased presence of a chemical or specific compound that is associated with a change or modulation in nerve activity. For example, Calcium or potassium sensors can be used. The electrodes can include standard electrodes, multi-electrode arrays (as shown in the figures) and expandable felt electrodes, all of which are well known to those of skill in the art. The soft, felt-like material of the felt electrode expands with moisture, maximizing contact with the larynx while reducing trauma and retains moisture to minimize impedance. Additionally, other sensors 14 can also be used without departing from the spirit of the present invention. The multi-electrode arrays 14′ can be formed of standard electrodes, felt electrodes, other electrodes and combinations thereof.

The sensors 14 can be attached or affixed to an exterior surface 13 of the cannula 12 at a location determined by those of skill in the art that will be in close proximity to the nerve to be monitored upon insertion of the cannula 12. The sensors 14 can be affixed directly to the exterior surface 13 of the cannula 12 via an adhesive or can be affixed to an affixing device that is placed about the ET tube. For example, the sensors 14 can be attached to a removable sleeve (not shown) that can be used as a retrofit for any currently available cannulas. The benefit of such a sleeve is that it can be adjustable and thus can be placed about any currently available cannula. Further, the sleeve eliminates the need for new cannulas to be manufactured, because the sleeve can be manufactured separately and affixed to the cannula 12 prior to insertion into the patient.

A sleeve could be placed over the ET tube 12′ prior or after intubation. The latter innovation would allow a conventional ET tube of normal diameter to be positioned followed by the sleeve that is slid over the ET tube thus acting as a stylet for the sleeve.

The sleeve can include pockets (not shown) into which the sensors 14 are placed. Alternatively, the sleeve can include sensor holding strips that maintain the sensors 14 in place on the exterior surface of the sleeve. The sensors 14 can either be integrated within the material of the sleeve or can be added post production thereby enabling the sensors to both be removed and be changed depending on the type of sensor needed.

As stated above, the sensors 14 can also be attached directly to the exterior surface of the cannula 12. In such a configuration the sensor can be attached via surgical or other adherence technique that enables attachment of the sensor 14 without altering the functionality of the sensor 14. For example, if the sensor is a chemical or compound sensor, it is important for the adhesive to not inhibit the function of the sensor.

The “alignment device” of the present invention is a device 16 capable of providing to the user an indication of the position of the sensors, assuring appropriate sensor location increases the accuracy of monitoring thereby limiting the risk of nerve damage. The alignment device 16 provides ongoing feedback to the user either as the receiver or the transmitter. The feedback can be in the form of a sound/alarm, a visual indicator, a vibration, electromagnetic energy or other form that provides electrode position status. The alignment device 16 is located on an insertion end 15 of the cannula 12.

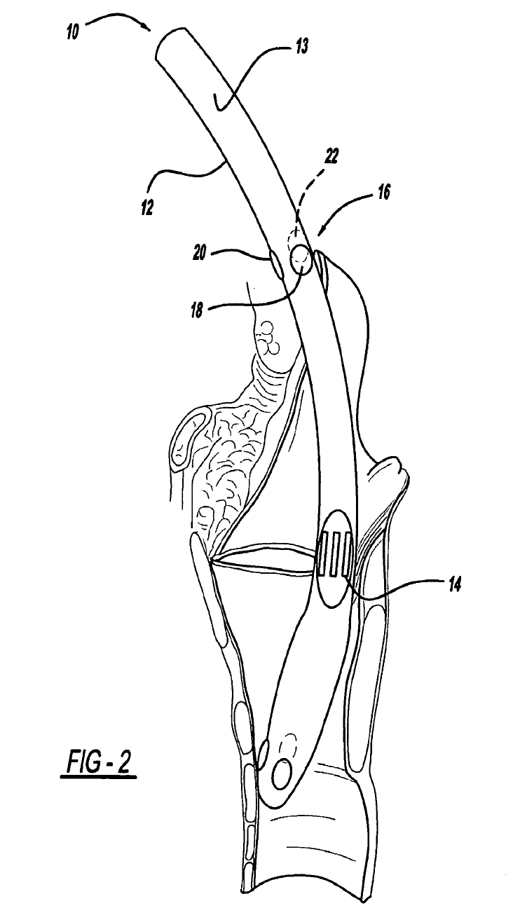

In one embodiment of the present invention, light emitting diodes 18,20,22 (LEDs) or other electromagnetic spectrum signals are included as part of the alignment device 16. Insulated wires connect the LEDs 18,20,22 to a power source that can include: a disposable battery, a re-usable and/or rechargeable battery-driven power source, a power source from the nerve monitoring apparatus, and an attachment that allows power from standard laryngoscopes to be used. The alignment device 16 can include indicators that provide readily understandable indications of whether the sensor is properly aligned. The indicators can be sound, a vibration, light or a display that is provided to the surgeon. For example, as shown in FIGS. 2A and 2B, color coded lights (LEDs 18,20,22) assist in determining ET tube position e.g. Red=Right, Blue=Left, Yellow=Midline.

Alternatively, the alignment device 16 can also include transillumination, such as fiberoptic illumination. In this type of illumination, fibers transmit light from an external source to illuminate the lateral and anterior borders of the ET tube, thereby indicating the position of the sensors.

There are numerous sources that can be used to power the LEDs 18,20,22: 1) a specially dedicated power source, 2) an attachment to nerve monitoring apparatus, and 3) a special attachment to the battery-powered laryngoscope used during intubation. Similarly, fiberoptic transillumination may be powered by numerous available light sources.

The embodiment shown in the figures combines the above components to maximize electrode-vocal cord contact while providing expedient feedback of ET tube 12′ position. The inventions may be used singly or in combination. The present invention solves the drawbacks of prior art. Modifications of an ET tube 12′ allow enhanced recording of laryngeal muscle response to mechanical and electrical stimulation. 1) A multi-channel electrode array 14′ allows monitoring from different areas of the glottis thereby compensating for inadvertent ET tube rotation; 2) Use of expandable felt-like electrodes allow improved contact and reduced impedance while diodes (LEDs) 18,20,22 or fiberoptic illumination allows assessment of ET tube 12′ position transcutaneously without the need for repeated endoscopy.

### Operation

In use, at least one sensor is attached to a cannula 12. The cannula 12 is selected based upon the specific use. The sensors enable the user, a doctor, to assess the location of the nerve to be monitored. The primary purpose being to protect the nerve from damage. However, it is possible that the sensor 14 can be used to detect the location of a nerve that is to be treated, and monitor the progress of a surgery or procedure designed to damage or render useless the nerve. After insertion, the alignment device 16 is used to ensure the sensor is properly located. The alignment device 16 can be turned off or kept on to ensure that the cannula 12 does not rotate during the surgery or procedure. The sensors 14 are then used to monitor nerve activity.

More specifically, a multi-channel electrode array 14′ is attached to an ET tube 12′, which allows monitoring from different areas of the glottis thereby compensating for inadvertent ET tube rotation and allowing multiple recording modalities. The uninsulated portions of the electrodes detect EMG responses from the laryngeal muscles. The insulated portions of the electrodes transmit the signal to the external EMG monitoring device. Unlike current available devices, which use flat metal electrodes, the multi-channel electrode array 14′ can utilize felt-like electrodes. After insertion into the patient, transillumination near the electrode array 14′ allows assessment of ET tube 12′ position transcutaneously without the need for repeated endoscopy (FIG. 2).

More specifically, immediately following intubation with a visual check of the ET tube 12′ position, the LEDs are connected to the power source. Appropriate ET tube 12′ position is determined by visualizing the transilluminated location of the LEDs 18,20,22 to assess correct depth and rotation of the ET tube 12′. The power source is then disconnected, to be used again if clinically indicated.

The eight color-coded electrodes from the multi-electrode array 14′ (four for the left and four for the right side) are connected to a nerve monitor with separate electrodes attached for ground and anode (stimulus return) on the sternum. Stimulating Dissectors or other nerve stimulators are then connected.

Impedances are tested and a tap test performed on the larynx to assure integrity of the set up. The initial stimulus intensity is set to 1 mA with alterations in the current based on clinical indications.

The multi-electrode array 14′ minimizes the deleterious effects of the ET tube rotation for the first time by allowing the surgeon or technician complete flexibility in choosing the optimal recording montage for each patient. Choices include 1) monitoring all channels, 2) monitoring selective channels based on impedance testing and responses to electrical stimulation, and 3) monitoring in monopolar or bipolar modalities.

The felt-like electrode tips can be moistened just prior to insertion or allowed to hydrate with the patients own secretions. In addition to the felt-like materials already used in surgery (e.g. brain cottonoids) soft, expandable materials such as Merocel® (Medtronic Xomed, Inc.), or other materials used for epistaxis and sinus surgery may be employed.

The above description is that of the current embodiment of the invention. Various alterations and changes can be made without departing from the spirit and broader aspects of the invention as defined in the appended claims, which are to be interpreted in accordance with the principles of patent law including the doctrine of equivalents. Any reference to a claim element in the singular, for example, using the articles “a,” “an,” “the” or “said,” is not to be construed as limiting the element to the singular.

It is to be understood that while we have illustrated and described certain forms of our invention, it is not to be limited to the specific forms or arrangements herein described and shown. The foregoing detailed description has been given for clearness of understanding only and no unnecessary limitations should be understood there from, as modifications will be obvious to those skilled in the art.

What is claimed:

1. A nerve monitoring device comprising:
a cannula;
at least one sensor for monitoring the nerve/muscle, said sensor being affixed to an exterior surface of said cannula; and
alignment means for ensuring proper alignment of said sensors for monitoring at least one nerve/muscle, said alignment means in communication with said sensor.

2. The device according to claim 1, wherein said sensor is an electrode.

3. The device according to claim 2, wherein said electrode is a felt-like electrode in a multi-electrode array.

4. The device according to claim 1, wherein said sensor is a multi-sensor array.

5. The device according to claim 1, wherein said cannula is an endotracheal tube.

6. The device according to claim 1, wherein said alignment means is a signal generated to indicate alignment of said sensors.

7. The device according to claim 6, wherein said signal is formed by a light emitting diode or other energy along the electromagnetic spectrum.

8. The device according to claim 1, further including a coating on an exterior surface of said cannula.

9. A method of monitoring nerve activity by:
inserting the nerve monitoring device according to claim 1 into a patient at a location in need of monitoring, thereby monitoring the nerve activity.

10. The method according to claim 9, further including the step of attaching the device to an external power source.

11. An endotracheal nerve monitoring device comprising:
an endotracheal tube;
at least one sensor for monitoring the nerve. said sensor being affixed to an exterior surface of said endotracheal tube; and
alignment means for ensuring proper alignment of said sensors for monitoring at least one nerve, said alignment means in communication with said sensor.

12. The device according to claim 1, wherein said sensor is an electrode.

13. The device according to claim 2, wherein said electrode is a felt-like, expandable electrode.

14. The device according to claim 1, wherein said sensor is a sensor array.

15. The device according to claim 1, wherein said alignment means is a signal generated to indicate alignment of said sensors.

16. The device according to claim 15, wherein said signal is formed by a light emitting diode or other energy along the electromagnetic spectrum.

17. The device according to claim 1, further including a coating on an exterior surface of said endotracheal tube.
